(19) United States
(12) Patent Application Publication
(10) Pub. No.: US 2005/0137408 A1
(43) Pub. Date: Jun. 23, 2005

(54) Process for producing aminoepoxide

(75) Inventors: Yasuyuki Otake, Kawasaki-shi (JP); Kunisuke Izawa, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC, Tokyo (JP)

(21) Appl. No.: 10/972,520

(22) Filed: Oct. 26, 2004

Related U.S. Application Data
(63) Continuation of application No. PCT/JP03/05069, filed on Apr. 21, 2003.

(30) Foreign Application Priority Data
Apr. 26, 2002 (JP) 2002-127579

(51) Int. Cl. C07D 301/24

(52) U.S. Cl. 549/520

(57) ABSTRACT

N-carbamate protected-3-amino-1,2-epoxy-4-(hydroxy substituted phenyl)butane may be produced in high optical purity and in high yield, by hydrogenating N-carbamate protected-3-amino-1-chloro-2-hydroxy-4-(benzyloxy substituted phenyl)butane in the presence of a metal catalyst to give N-carbamate protected-3-amino-1-chloro-2-hydroxy-4-(hydroxy substituted phenyl)butane and treating this compound with a base.

### CROSS REFERENCES TO RELATED APPLICATIONS

This application is a continuation of International Patent Application No. PCT/JP03/05069, filed on Apr. 21, 2003, and claims priority to Japanese Patent Application No. 2002-127579, filed on Apr. 26, 2002, both of which are incorporated herein by reference in their entireties.

### BACKGROUND OF THE INVENTION

1. Field of the Invention

The present invention relates to a method for producing optically active aminoepoxides represented by the following formula (1), which are useful as an intermediate compounds for pharmaceutical products such as an HIV protease inhibitors and the like.

2. Discussion of the Background

Aminoepoxide compounds represented by formula (1) 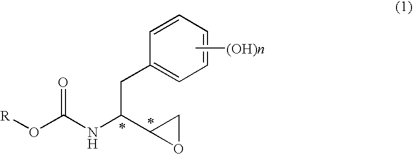
wherein R is an alkyl group or a fluorenylmethyl group, n is an integer of 1 or 2 and * indicates an asymmetric carbon atom independently having an R or S configuration, are known to be useful as intermediate compounds for an HIV protease inhibitor (e.g., P. Chen et al., Journal of Medicinal Chemistry, vol. 39, no. 10, p. 1991 (1996)).

Methods of producing aminoepoxide compounds represented by formula (1), depicted in the following Scheme 1, are disclosed in Journal of Organic Chemistry vol. 61, no. 11, p. 3635 (1996)) and WO99/10373. 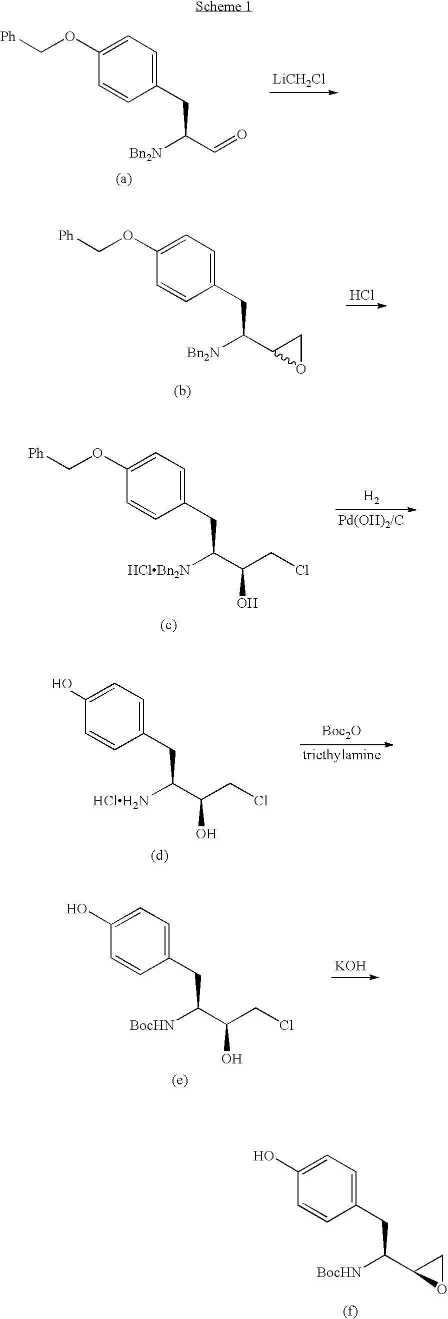
wherein Ph is a phenyl group, Bn is a benzyl group, and Boc is a tert-butoxycarbonyl group.

The disclosed method comprises removing two benzyl groups, which is an amino-protecting group, from the amino group of compound (c), while simultaneously removing a benzyl group protecting from a hydroxyl group, and again protecting the amino group with a carbamate protecting group. To stereoselectively obtain compound (c) having a (2S,3S) or (2R,3R) configuration as in the disclosed method, the amino group is protected with a two benzyl groups and an aldehyde compound (a) is used as a starting material. However, since the aldehyde compound (a) is relatively easily racemized, close control of the production conditions and step operations are required. Therefore, this method is not entirely preferable for industrial production. Moreover, as mentioned above, a step for converting the amino-protecting group is necessary. Furthermore, in the aforementioned reference J. Med. Chem., vol. 39, p. 1996 (1991), the yield of aminoepoxide compound (f) is reported to be 93% by the reaction after deprotection of compound (c). In WO99/10373, however, the yield is reported as not more than 50% by the same method.

Even when a debenzylation reaction of hydroxyl group is carried out under the conditions described in this reference and using a compound wherein the amino group is protected by a tert-butoxycarbonyl group instead of the dibenzyl group of compound (c), deprotection (decarbamation) of the amino group proceeds due to the acidic conditions and a step for protecting the amino group again becomes necessary.

A different production method of aminoepoxide compound (f) is disclosed in Journal of Medicinal Chemistry vol. 39, no. 10, p. 1991 (1996), as shown in Scheme 2. 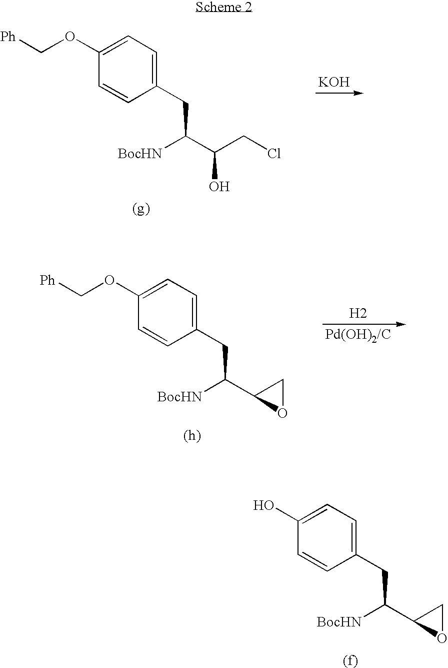
wherein Ph is a phenyl group and Boc is a tert-butoxycarbonyl group.

In Journal of Medicinal Chemistry, vol. 39, no. 10, p. 1991 (1996), the epoxide compound (h) was isolated and purified twice. In fact, when the debenzylation reaction was carried out without isolation and purification of compound (h), side reaction proceeded due to the impurity produced in the system and a drastic degradation of quality and yield was confirmed. Since this kind of epoxide shows mutagenicity, isolation and purification of epoxide compound (h) require special facility. Therefore, this method is also hardly suitable for industrialization.

In addition, WO99/10373 discloses a production method shown in Scheme 3. 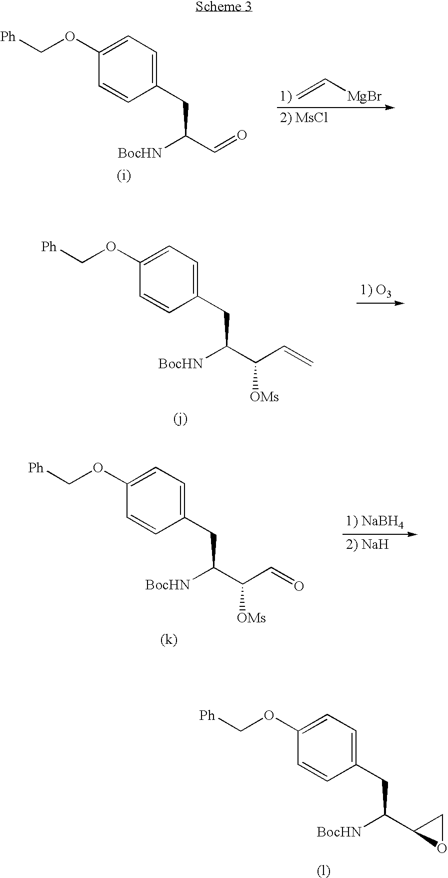
wherein Ph is a phenyl group, Boc is a tert-butoxycarbonyl group, and Ms is a mesyl group.

Since the above-mentioned production method also invlves the use of a more optically unstable aldehyde compound (i) than the aforementioned aldehyde compound (a) and includes a step of ozone oxidization, which is difficult to industrialize, this method is also not entirely suitable for industrialization.

Thus, there remains a need for a method of producing aminoepoxides represented by formula (1) with a high optical purity and in a high yield.

### SUMMARY OF THE INVENTION

Accordingly, it is one object of the present invention to provide novel methods for producing a compound of formula (1).

It is another object of the present invention to provide novel methods for producing a compound of formula (1) with a high optical purity.

It is another object of the present invention to provide novel methods for producing a compound of formula (1) in a high yield.

It is another object of the present invention to provide novel methods for producing a compound of formula (1) with a high optical purity and in a high yield.

These and other objects, which will become apparent during the following detailed description, have been achieved by the inventors' discovery that hydrogenation of N-carbamate protected-3-amino-1-chloro-2-hydroxy-4-(benzyloxy-substituted phenyl)butane (the compound represented by the following formula (2)) in the presence of a metal catalyst affords N-carbamate protected-3-amino-1-chloro-2-hydroxy-4-(hydroxy-substituted phenyl)butane (the chlorohydrin compound represented by the following formula (3)), without confirmed elimination of chlorine by the reduction reaction. Furthermore, they have found that treatment of the obtained N-carbamate protected-3-amino-1-chloro-2-hydroxy-4-(hydroxy-substituted phenyl)butane with a base affords a highly optically pure N-carbamate protected-3-amino-1,2-epoxy-4-(hydroxy-substituted phenyl)butane (the aminoepoxide compound represented by the following formula (1)) in a high yield.

Accordingly, the present invention provides the following:
1. A method for producing an aminoepoxide compound represented by formula (1): 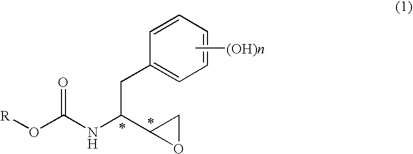
   wherein R is an alkyl group or a fluorenylmethyl group, n is an integer of 1 or 2, and * indicates an asymmetric carbon atom independently having an R or S configuration, which comprises:
(a) hydrogenating a compound represented by formula (2): 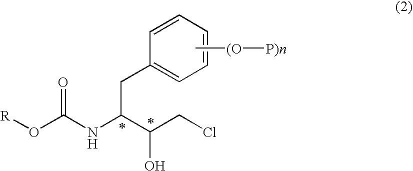
   wherein R, n, and * are as defined above and P is a benzyl group optionally having one or more substituents, in the presence of a metal catalyst to give a chlorohydrin compound represented by formula (3): 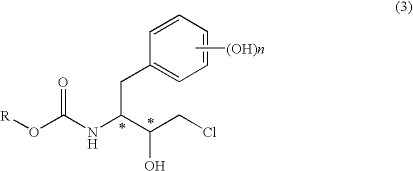
   wherein R, n, and * are as defined above; and
(b) treating the chlorohydrin compound of formula (3) with a base, to obtain the aminoepoxide compound represented by formula (1).
2. The method of 1, wherein configurations of the 2-position and the 3-position of the aminoepoxide compound represented by formula (1), the compound represented by formula (2) and the chlorohydrin compound represented by formula (3) are each an S configuration or an R configuration.
3. The method of 1 or 2, wherein R is a tert-butoxycarbonyl group, n is 1, and the position of substitution on the aromatic ring is the 4-position.

When the configuration of the (2-position and 3-position) of an aminoepoxide compound represented by formula (1) is (2S,3S), (2R,3R), (2R,3S) or (2S,3R), the configuration of the (2-position and 3-position) of a corresponding compound represented by formula (2) is consequently (2S,3S), (2R,3R), (2R,3S) or (2S,3R), and the configuration of the (2-position and 3-position) of a corresponding chlorohydrin compound represented by formula (3) is consequently (2S,3S), (2R,3R), (2R,3S) or (2S,3R).

The method of the present invention is particularly preferably applied when the configurations of the 2-position and the 3-position of an aminoepoxide compound represented by formula (1), a compound represented by formula (2) and a chlorohydrin compound represented by formula (3) are each an S configuration (i.e., (2S,3S)), or an R configuration (i.e., (2R,3R)).

### DETAILED DESCRIPTION OF THE PREFERRED EMBODIMENTS

Thus, in a first embodiment, the present invention provides novel for producing an aminoepoxide compound represented by formula (1): 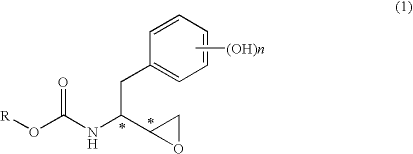
wherein R is an alkyl group or a fluorenylmethyl group, n is an integer of 1 or 2, and * indicates an asymmetric carbon atom independently having an R or S configuration, which comprises:
(a) hydrogenating a compound represented by formula (2): 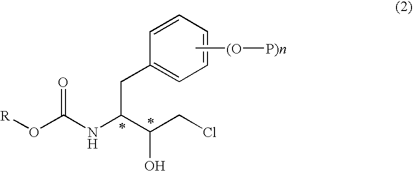
   wherein R, n, and * are as defined above and P is a benzyl group optionally having one or more substituents, in the presence of a metal catalyst to give a chlorohydrin compound represented by formula (3): 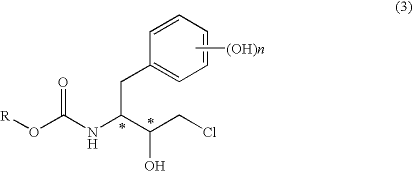
   wherein R, n, and * are as defined above; and
(b) treating the chlorohydrin compound of formula (3) with a base, to obtain the aminoepoxide compound represented by formula (1).

In formulae (1), (2), and (3), n is an integer of 1 or 2. That is, the compound represented by formula (1) or formula (3) is a compound having 1 or 2 hydroxyl groups on the phenyl group, and a compound represented by formula (2) is a compound having 1 or 2 protected hydroxyl groups on the phenyl group. It is particularly preferred that the compounds represented by formula (1) or formula (3) in the present invention are compounds having a 4-hydroxyphenyl group (ie., compounds wherein n=1 and the position of substitution on the aromatic ring is the 4-position, ie., para-substitution).

In formula (2), n is an integer of 1 or 2 and P is a benzyl group optionally having one or more substituents. In other words, a compound represented by the formula (2) has 1 or 2 benzyloxy groups (in which the aromatic ring of each benzyloxy group optionally has one or more substituents) on the phenyl group.

The substituent which may be present on P is not particularly limited as long as it is a group that does not exert an adverse influence on the reactions in the present invention and, for example, an alkoxy group (preferably having 1 to 7 carbon atoms), an alkyl group (preferably having 1 to 7 carbon atoms), a nitro group, a halogen group and the like can be mentioned. Generally, a compound in which P is a benzyl group is preferably used. A compound represented by formula (2), which is particularly preferably used in the present invention, is a compound containing a 4-benzyloxyphenyl group (namely, n=1, P=benzyl group, and the position of substitution on the aromatic ring is the 4-position, i.e., para-substitution).

In formulae (1)-(3) of the present invention, R is an alkyl group or a fluorenylmethyl group. The alkyl group has 1 to 10 carbon atoms, and is preferably an alkyl group having 1 to 4 carbon atoms. For example, a methyl group, an ethyl group, a tert-butyl group and the like can be mentioned.

The compounds represented by formula (2), which are used as starting materials in the present invention, are known compounds, and can be produced by known methods such as reduction of a chloroketone compound represented by formula (4) and the like (see WO0/44706, Europe Patent EP 1 081 133). 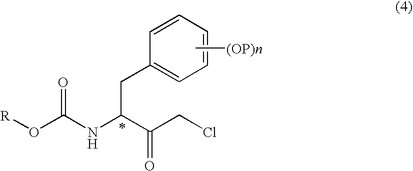
wherein R, P, and n are as defined above, * indicates an asymmetric carbon atom, which means the denoted carbon atom independently has an R or S configuration.

In the following, the method of producing a chlorohydrin compound of formula (3), which comprises hydrogenation (debenzylation) of a compound of formula (2) in the presence of a metal catalyst, is explained in detail.

As the metal catalyst, for example, palladium hydroxide on carbon, palladium carbon, Lindlar's catalyst, and the like can be mentioned. The use of palladium hydroxide on carbon is preferred.

The amount of the metal catalyst used is not particularly limited and is preferably 0.0005-0.2 equivalent, based on the number of moles of the compound of formula (2).

Any hydrogen source which is generally used for debenzylation, such as hydrogen gas, cyclohexadiene, formic acid ammonium salt and the like, can be used as the hydrogen source to be coexistent in the reaction system. Hydrogen gas and cyclohexadiene are preferred, and hydrogen gas is particularly preferred.

The hydrogen pressure when hydrogen gas is used as the hydrogen source is preferably 1-10 atm, and particularly preferably 1-1.5 atm.

As the reaction solvent, for example, protic solvents such as methanol, ethanol, 1-propanol, 2-propanol, 1-butanol, water, and the like; and aprotic solvents such as ethyl acetate, isopropyl acetate, acetone, 2-butanone, methylisobutylketone, tetrahydrofuran, 1,4-dioxane, toluene, and the like can be preferably used. When the hydrogenation step and the following epoxidation reaction are to be conducted successively, the preferred solvent to be used is 2-propanol or a mixed solvent of 2-propanol and water, which can be preferably used for both steps. When a mixed solvent is used, the volume ratio of 2-propanol and water is preferably in the range of 2-propanol:water=1:1 to 100:1, more preferably in the range of 1:1 to 50:1. These reaction solvents may be used alone or in combination of one or more kinds thereof.

For the hydrogenation, a base may be added to the reaction mixture to increase the reaction rate. Suitable such bases include, for example, lithium hydroxide, sodium hydroxide, potassium hydroxide, sodium carbonate, potassium carbonate, sodium hydrogen carbonate, potassium hydrogen carbonate, and the like. Particularly preferred is sodium hydrogen carbonate. These bases may be added to the reaction mixture in the form of a solid, or may be used as an aqueous solution. While the amount of the base to be used varies depending on the identity of the base to be used, when the amount of the base is too large, the dechlorination reaction tends to proceed, and therefore, the amount is preferably not more than 1 equivalent, particularly preferably not more than 0.3 equivalent, relative to the amount of the compound represented by formula (2) used.

While the reaction temperature also varies depending on the kind of solvent and the amount of the base used, it is generally 20° C. to 80° C., preferably 40° C. to 60° C. The reaction temperature may be changed during the course of the reaction. While the reaction time is not particularly limited, it is preferably about 30 min to 24 hr.

The reaction is generally conducted with stirring. After the completion of the reaction, the metal catalyst is removed from the reaction mixture by filtration and the like to give a solution containing a chlorohydrin compound represented by formula (3). In this case, the chlorohydrin compound represented by formula (3) can be continuously subjected to an epoxidation reaction in the next step, without isolation.

The solubility of the chlorohydrin compound represented by formula (3) in a reaction mixture can be increased by basifying the reaction solution by adding a base before removing the metal catalyst by filtration. When a metal catalyst is removed by filtration, therefore, the chlorohydrin compound can be prevented from being precipitated and the catalyst simultaneously removed by filtration. In this case, the chlorohydrin compound is partially or entirely converted to aminoepoxide represented by formula (1). As the base to be used, aqueous lithium hydroxide solution, aqueous sodium hydroxide solution, aqueous potassium hydroxide solution, aqueous sodium carbonate solution, aqueous potassium carbonate solution, aqueous sodium hydrogen carbonate solution, aqueous potassium hydrogen carbonate solution, and the like can be mentioned. Particularly preferred is aqueous sodium hydroxide solution. The amount of base to be used is preferably 0.1 to 5.0 equivalents, particularly preferably 0.1 to 3.0 equivalents, relative to the amount of the compound represented by formula (2) used in the hydrogenation step. When a base has been already used in the hydrogenation step, the difference between the above-mentioned amount of the base and the amount of the base already used can be added.

To increase the yield, the metal catalyst removed by filtration may be washed with a mixed solvent of an organic solvent and a base and the washing liquid may be added to the above-mentioned solution.

As the organic solvent to be used for washing the metal catalyst, protic solvents such as methanol, ethanol, 1-propanol, 2-propanol, 1-butanol, water, and the like; or aprotic solvents such as ethyl acetate, isopropyl acetate, acetone, 2-butanone, methyl isobutyl ketone, tetrahydrofuran, 1,4-dioxane, toluene, and the like can be preferably used. Particularly preferred is 2-propanol.

As a base to be mixed with an organic solvent for washing the metal catalyst, aqueous lithium hydroxide solution, aqueous sodium hydroxide solution, aqueous potassium hydroxide solution, aqueous sodium carbonate solution, aqueous potassium carbonate solution, aqueous sodium hydrogen carbonate solution, aqueous potassium hydrogen carbonate solution, and the like can be mentioned. Particularly preferred is aqueous sodium hydroxide solution. The amount of the base to be used including the amount of a base already used is preferably within the range not exceeding 5.0 equivalents, particularly preferably 3.0 equivalents, relative to the amount of the compound represented by formula (2).

The chlorohydrin compound represented by the formula (3) can be continuously subjected to the epoxidation reaction in the next step without isolation. As a reaction solvent in this case, 2-propanol or a mixed solvent of 2-propanol and water, which can be also preferably used in the hydrogenation step, can be used.

The method of obtaining an aminoepoxide compound represented by formula (1) by reacting a base with a chlorohydrin compound represented by formula (3) is now explained in detail.

While the organic solvent to be used for the reaction is not particularly limited, as long as it does not exert an adverse influence on the reaction, for example, methanol, ethanol, 1-propanol, 2-propanol, 1-butanol, water, and the like can be preferably used. Particularly, 2-propanol and a mixed solvent of 2-propanol and water are used. These reaction solvents may be used alone or in combination of one or more kinds thereof. As the base to be used, aqueous lithium hydroxide solution, aqueous sodium hydroxide solution, aqueous potassium hydroxide solution, aqueous sodium carbonate solution, aqueous potassium carbonate solution, aqueous sodium hydrogen carbonate solution, aqueous potassium hydrogen carbonate solution, and the like can be mentioned. Particularly preferred is aqueous sodium hydroxide solution.

The amount of the base to be used is generally 2.0 to 5.0 equivalents, preferably 2.0 to 3.0 equivalents, based on the amount of the compound of formula (3). When a base has been already added in a hydrogenation step, a step of removing a metal catalyst by filtration, and the like, a base can be used in the above-mentioned amount less the amount of the base already used.

After the completion of the reaction, any excess base is neutralized with an acid. As the acid to be used here, for example, hydrochloric acid, sulfuric acid, nitric acid, acetic acid, citric acid, and the like can be mentioned. Particularly preferred is citric acid. While the amount of the acid to be used varies depending on the amount of the base to be used, for example, it is preferably 1.0 to 3.0 equivalents when the amount of the base to be used is 2.0 to 3.0 equivalents, each based on the number of moles of the compound of formula (3).

The reaction is generally carried out with stirring, and the reaction temperature is generally preferably −10° C. to 40° C., particularly preferably 0° C. to 25° C. The reaction temperature may be changed during the course of the reaction. While the reaction time is not particularly limited, it is preferably about 30 min to 24 hr.

The reaction mixture is crystallized by concentration as necessary, or the solvent is completely evaporated from the reaction mixture as necessary and a different solvent is used to conduct crystallization to give the aminoepoxide represented by formula (1) as crystals.

When the configuration is an S configuration (i.e., (2S,3S)) or an R configuration (i.e., (2R,3R) for both the 2-position and the 3-position, the solvent to be used for the crystallization is preferably a mixed solvent of a protic solvent such as methanol, ethanol, 1-propanol, 2-propanol, 1-butanol, and the like and water, and a mixed solvent of 2-propanol and water is particularly preferable. As the temperature of crystallization, −10° C. to 20° C. is preferable. For example, when these solvents have been used as a reaction solvent for the epoxidation reaction, crystallization can be performed directly (or after concentration as necessary).

In a preferred embodiment, the starting material of formula (2) and the product of formula (1) are diastereomerically enriched or pure. By diastereomerically enriched, it is meant that one pair of enantiomers is present in a greater amount than the other pair of enantiomers. In this context, it is to be understood that the optical isomers (2S,3S) and (2R,3R) form one enantiomeric pair, while the optical isomers (2S,3R) and (2R,3S) form the other pair of enantiomers. It is preferred that the product of formula (1) have a diastereomeric excess, de, of at least 50%, more preferably at least 75%, even more preferably at least 90%, still more preferably at least 95%, even still more preferably at least 97%. In this context, the diastereomeric excess for an enantiomer pair P₁ is calculated as follows:
de=(P₁−P₂)÷(P₁+P₂)
   where P₁ and P₂ are the amounts of the two diastereomeric pairs in a mixture.

In another preferred embodiment, the starting material of formula (2) and the product of formula (1) are enantiomerically enriched or pure. By enantiomerically enriched, it is meant that one enantiomer is present in a greater amount than the other enantiomer of the same enantiomeric pair. As noted above, the optical isomers (2S,3S) and (2R,3R) form one enantiomeric pair, while the optical isomers (2S,3R) and (2R,3S) form the other pair of enantiomers. It is preferred that the product of formula (1) have a enantiomeric excess, ee, of at least 50%, more preferably at least 75%, even more preferably at least 90%, still more preferably at least 95%, even still more preferably at least 97%, more preferably yet at least 99%. In this context, the enantiomeric excess for an enantiomer E₁ is calculated as follows:
ee=(E₁−E₂)÷(E₁+E₂)
where E₁ and E₂ are the amounts of the two enantiomers in a mixture.

The aminoepoxide compound represented by formula (1) obtained in this way can be also subjected to protection of one or both of the hydroxyl groups on the aromatic ring with a suitable protecting group as necessary.

Since the series of reactions explained above proceeds stereoselectively, the object compound can be obtained in a high optical purity, as well as in a high yield. As one example of a preferable embodiment of the method of the present invention, a series of reaction steps for producing (2S,3S)-N-carbamate protected-3-amino-1,2-epoxy-4-(4-hydroxyphenyl)butane by using (2S,3S)-N-carbamate protected-3-amino-1-chloro-2-hydroxy-4-(4-benzyloxyphenyl)butane are shown in the following Scheme 4. 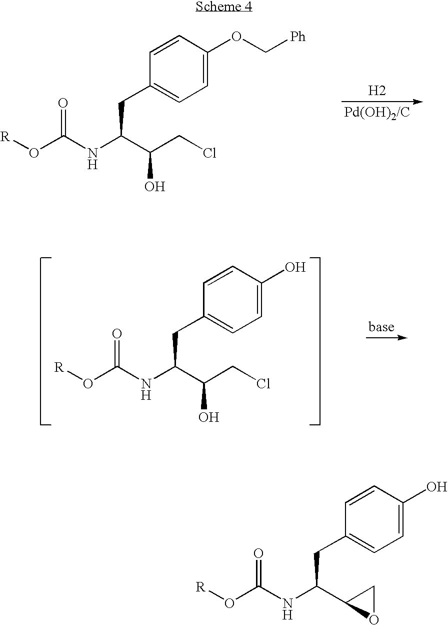
wherein R is as defined above. In one example of a preferred embodiment R is a tert-butyl group.

According to the present invention, aminoepoxides represented by the above-mentioned formula (1) can be produced with a high optical purity and in a high yield.

Other features of the invention will become apparent in the course of the following descriptions of exemplary embodiments which are given for illustration of the invention and are not intended to be limiting thereof.

### EXAMPLES

In the following examples, all temperatures are in degrees Celsius, unless expressly stated to be otherwise.

### Reference Example 1

### Production of Methyl 2R-amino-3-(4-benzyloxyphenyl)propionate Hydrochloride

1N Aqueous sodium hydroxide solution (26.2 ml) and copper sulfate (2.09 g) were added to 2R-amino-3-(4-hydroxyphenyl)propionic acid (4.75 g), and the mixture was stirred at room temperature for 1 hr. To this solution were added methanol (158 ml) and 2N aqueous sodium hydroxide solution (13.1 ml), and then benzyl bromide (3.3 ml) was added slowly. The mixture was stirred at room temperature for 3 hr. The product was collected by filtration and the crystals were washed with a mixed solvent of methanol (12.5 ml) and water (12.5 ml). 1N Aqueous hydrochloric acid solution (30 ml) was added to the obtained crystals and the mixture was stirred at 50° C. for 30 min. The crystals were collected by filtration and 1N aqueous hydrochloric acid solution (30 ml) was added to the obtained crystals. The mixture was stirred at 50° C. for 30 min, and the crystals were collected by filtration. The obtained crystals were washed with water (25 ml), after which water (30 ml) was added. The mixture was adjusted to pH 6.3 with sodium hydroxide, and the crystals were collected by filtration. The obtained crystals were dried to give 2R-amino-3-(4-benzyloxyphenyl)propionic acid as crystals (3.24 g, yield 46%). Thionyl chloride (0.959 ml) was added to methanol (32.4 ml) at 0° C., and the obtained crystals (3.24 g) of 2R-amino-3-(4-benzyloxyphenyl)propionic acid were added to this solution. The mixture was stirred at 60° C. for 15 hr. This reaction mixture was concentrated under reduced pressure, and ethyl acetate (65 ml) was added to allow crystallization. The obtained crystals were collected by filtration and dried under reduced pressure to give the object methyl 2R-amino-3-(4-benzyloxyphenyl)propionate hydrochloride (3.30 g, yield by crystallization 97%, yield relative to 2R-amino-3-(4-hydroxyphenyl)propionic acid 44%).

¹H-NMR (CD₃OD, 400 MHz) 8 ppm: 3.12 (dd, 1H, J=14.4, 7.3 Hz), 3.19 (dd, 1H,J=14.4, 6.0 Hz), 3.79 (s, 3H), 4.27 (dd, 1H, J=7.4, 6.0 Hz), 4.85 (s, 3H), 5.08 (s, 2H), 6.98-7.01 (m, 2H), 7.16-7.23 (m, 2H), 7.26-7.45 (m, 5H)

¹³C-NMR (CD₃OD, 400 MHz) δppm: 37.0, 54.0, 55.7, 71.4, 117.0, 127.7, 129.0, 129.3, 129.9, 132.0, 139.0, 160.3, 170.9

mass spectrum m/e: 286 (M-Cl)⁺

### Reference Example 2

### Production of Methyl 2S-N-phenylmethylene-2-amino-3-(4-benzyloxyphenyl)propionate

To methyl 2S-amino-3-(4-benzyloxyphenyl)propionate hydrochloride (5.15 g, manufactured by KOKUSAN CHEMICAL Co., Ltd.) was added dichloromethane (25.8 ml) under an argon atmosphere, and the mixture was stirred at 0° C. Sodium sulfate (4.55 g), benzaldehyde (1.63 ml), and triethylamine (2.23 ml) were added, and the mixture was stirred at 0° C. for 23 hr. Sodium sulfate was removed by filtration, and the obtained solution was concentrated under reduced pressure. t-Butyl methyl ether (50 ml) was added, and insoluble materials were removed by filtration. The obtained solution was concentrated under reduced pressure to give the object methyl 2S-N-phenylmethylene-2-amino-3-(4-benzyloxyphenyl)propionate (5.16 g, yield 86%).

¹H-NMR (CDCl₃, 400 MHz) δppm: 3.08 (dd, 1H, J=13.6, 8.0 Hz), 3.30 (dd, 1H,J=13.6, 5.2 Hz), 3.74 (s, 3H), 4.13 (dd, 1H, J=8.8, 5.2 Hz), 4.99 (s, 2H), 6.83-7.72 (m, 14H), 7.92 (s, 1H)

¹³C-NMR (CDCl₃, 400 MHz)₆ ppm: 39.4, 52.6, 70.4, 75.7, 115.1, 115.4, 127.9, 128.3, 128.9, 128.9, 130.1, 131.2, 131.5, 136.0, 137.5, 157.9, 164.1, 172.6

mass spectrum m/e: 374 (M+H)⁺

### Reference Example 3

### Production of methyl 2R-N-phenylmethylene-2-amino-3-(4-benzyloxyphenyl)propionate

To methyl 2R-amino-3-(4-benzyloxyphenyl)propionate hydrochloride (3.20 g) obtained in Reference Example 1 was added dichloromethane (8.64 ml) under an argon atmosphere, and the mixture was stirred at 5° C. Sodium sulfate (2.83 g), benzaldehyde (1.01 ml), and triethylamine (1.39 ml) were added, and the mixture was stirred at 5° C. for 18 hr. Sodium sulfate was removed by filtration, and the obtained solution was concentrated under reduced pressure. t-Butyl methyl ether (31 ml) was added, and insoluble materials were removed by filtration. Furthermore, t-butyl methyl ether (31 ml) was added, and insoluble materials were removed by filtration. The obtained solution was concentrated under reduced pressure to give the object methyl 2S-N-phenylmethylene-2-amino-3-(4-benzyloxyphenyl)propionate (3.01 g, yield 82%).

### Reference Example 4

### Production of 3S-amino-1-chloro-4-(4-benzyloxyphenyl)-2-butanone Hydrochloride

To methyl 2S-N-phenylmethylene-2-amino-3-(4-benzyloxyphenyl)propionate (4.0 g) obtained in the same manner as in Reference Example 2 were added bromochloromethane (0.905 ml), tetrahydrofuran (15 ml), and toluene (15 ml), and the mixture was stirred under an argon atmosphere at −78° C. A solution (2.66 M, 5.23 ml) of normal butyl lithium in hexane was slowly added dropwise over 1.5 hr. After the completion of the dropwise addition, the mixture was further stirred for 30 min, and the reaction mixture was poured at once into a mixed solution of 36% aqueous hydrochloric acid solution (2.39 ml) and methanol (5.40 ml) to quench the reaction. This solution was stirred at 25° C. for 1 hr, and the resulting crystals were collected by filtration. The obtained crystals were washed with hexane (10 ml) and dried under reduced pressure to give 3S-amino-1-chloro-4-(4-benzyloxyphenyl)-2-butanone hydrochloride (2.65 g, yield 73%).

¹H-NMR (DMSO-d₆,400 MHz) δppm: 2.84-3.14 (m, 2H), 4.43-4.50 (m, 1H), 4.52 (d, 1H, J=17.4 Hz), 4.74 (d, 1H, J=17.4 Hz), 5.08 (s, 2H), 6.94-7.00 (m, 2H), 7.21-7.25 (m, 2H), 7.30-7.53 (m, 5H), 8.73 (bs, 3H)

¹³C-NMR (DMSO-d₆, 400 MHz) δppm: 34.5, 48.3, 57.7, 69.5, 115.0, 126.8, 128.0, 128.2, 128.8, 131.0, 137.4, 158.0, 198.6

mass spectrum m/e: 304 (M-Cl)⁺

### Reference Example 5

### Production of 3R-amino-1-chloro-4-(4-benzyloxyphenyl)-2-butanone Hydrochloride

To methyl 2R-N-phenylmethylene-2-amino-3-(4-benzyloxyphenyl)propionate (3.0 g) obtained in the same manner as in Reference Example 3 were added bromochloromethane (0.680 ml), tetrahydrofuran (11.3 ml), and toluene (11.3 ml), and the mixture was stirred under an argon atmosphere at −78° C. A solution (2.66 M, 3.92 ml) of normal butyl lithium in hexane was slowly added dropwise over 2 hr. After the completion of the dropwise addition, the mixture was further stirred for 30 min, and the reaction mixture was poured at once into a mixed solution of 36% aqueous hydrochloric acid solution (1.79 ml) and methanol (4.05 ml) to quench the reaction. This solution was stirred at 25° C. for 1 hr and at 0° C. for 1 hr. The resulting crystals were collected by filtration and washed with hexane (7.5 ml). The crystals were dried under reduced pressure to give 3R-amino-1-chloro-4-(4-benzyloxyphenyl)-2-butanone hydrochloride (2.65 g, yield 97%).

### Reference Example 6

### Production of (2S,3S)-N-tert-butoxycarbonyl-3-amino-4-(4-benzyloxyphenyl)-1-chloro-2-hydroxybutane

di-tert-Butoxycarbonate (10.4 g), potassium carbonate (6.58 g), dichloromethane (135 ml), and water (67.5 ml) were stirred at 35° C., and 3S-amino-1-chloro-4-(4-benzyloxyphenyl)-2-butanone hydrochloride (13.5 g) obtained in the same manner as in Reference Example 4 was added. This reaction mixture was stirred at 35° C. for 2 hr. The organic layer and the aqueous layer were separated, and the organic layer was washed with 1N aqueous hydrochloric acid solution (54 ml) and water (54 ml). The obtained organic layer was concentrated under reduced pressure, and ethanol (30 ml) was added. The mixture was concentrated again under reduced pressure. Ethanol (144 ml) was added, and sodium borohydride (0.647 g) was added in several portions at room temperature. After stirring at room temperature for 2 hr, sodium borohydride (0.065 g) was further added, and the mixture was stirred at room temperature for 1 hr. The reaction was quenched using acetic acid (1.12 ml), and the reaction mixture was heated to 70° C. This solution was allowed to cool to 20° C. over 5 hr, and the mixture was stirred at 20° C. for 10 hr. The obtained crystals were collected by filtration, and the crystals were washed with ethanol (22 ml). The crystals were further washed twice with water (45 ml, 45 ml). The obtained crystals were dried under reduced pressure to give the object (2S,3S)-N-tert-butoxycarbonyl-3-amino-4-(4-benzyloxyphenyl)-1-chloro-2-hydroxybutane (9.50 g, yield 59%). The diastereoselectivity then was (2S,3S)/(2R,3S)=98.9/1.1.

¹H-NMR (DMSO-d₆,400 MHz) δppm: 1.28 (s, 9H), 2.88-2.47 (m, 1H), 3.35-3.70 (m, 5H), 5.05 (s, 2H), 5.41 (d, 1H, J=6.0 Hz), 6.65 (d, 1H, J=8.4 Hz), 6.89 (d, 2H, J=8.8 Hz), 7.08 (d, 2H, J=8.8 Hz), 7.30-7.45 (m, 5H)

¹³C-NMR (DMSO-d₆, 400 MHz) δppm: 28.5, 35.0, 48.4, 54.9, 69.5, 73.1, 77.9, 114.7, 127.9, 128.1, 128.7, 130.5, 131.6, 137.7, 155.5, 157.0

mass spectrum m/e: 406 (M+H)⁺

[α]²⁰_{D}=7.0° (c=1.0, CH₂Cl₂)

melting point: 161-162° C.

### Reference Example 7

### Production of (2R,3R)-N-tert-butoxycarbonyl-3-amino-4-(4-benzyloxyphenyl)-1-chloro-2-hydroxybutane

di-tert-Butoxycarbonate (1.92 g), potassium carbonate (1.22 g), dichloromethane (25 ml), and water (12.5 ml) were stirred at 35° C., and 3R-amino-1-chloro-4-(4-benzyloxyphenyl)-2-butanone hydrochloride (2.50 g) obtained in the same manner as in Reference Example 5 was added. This reaction mixture was stirred at 35° C. for 1.5 hr. The organic layer and the aqueous layer were separated, and the organic layer was washed with 1N aqueous hydrochloric acid solution (10 ml) and water (10 ml). The obtained organic layer was concentrated under reduced pressure, and ethanol (5.5 ml) was added. The mixture was concentrated again under reduced pressure. Ethanol (27.2 ml) was added, and sodium borohydride (0.128 g) was added in several portions at room temperature. After stirring at 25° C. for 1 hr, the reaction was quenched using acetic acid (0.193 ml), and the reaction mixture was heated to 70° C. This solution was allowed to cool to 20° C. over 5 hr, and the mixture was stirred at 20° C. for 12 hr. The obtained crystals were collected by filtration, and the crystals were washed with ethanol (4.1 ml). The crystals were further washed twice with water (8.2 ml, 8.2 ml). The obtained crystals were dried under reduced pressure to give the object (2R,3R)-N-tert-butoxycarbonyl-3-amino-4-(4-benzyloxyphenyl)-1-chloro-2-hydroxybutane (1.80 g, yield 60%). The diastereoselectivity then was (2R,3R)/(2S,3R)=99.0/1.0.

### Example 1

### Production of (2S,3S)-N-tert-butoxycarbonyl-3-amino-4-(4-hydroxyphenyl)-1-chloro-2-hydroxybutane

Ethanol (2.5 ml) was added to (2S,3S)-N-tert-butoxycarbonyl-3-amino-4-(4-benzyloxyphenyl)-1-chloro-2-hydroxybutane (0.250 g) obtained in the same manner as in Reference Example 6.20% palladium hydroxide on carbon (0.0025 g, manufactured by Kawaken Fine Chemicals Co., Ltd., the moisture content 49.1%) was further added under an argon atmosphere, and the mixture was stirred under a hydrogen atmosphere (1 atm) at 25° C. for 3 hr. After stirring further at 40° C. for 3 hr, palladium hydroxide on carbon was removed by filtration. The obtained solution was concentrated under reduced pressure to give the object (2S,3S)-N-tert-butoxycarbonyl-3-amino-4-(4-hydroxyphenyl)-1-chloro-2-hydroxybutane (0.191 g, yield 98%).

¹H-NMR (DMSO-d₆, 400 MHz) δppm: 1.29 (s, 9H), 2.46 (dd, 1H, J=14.0, 9.6 Hz), 2.83-2.92 (m, 1H), 3.30-3.65 (m, 5H), 6.62-6.68 (m, 3H), 6.94 (d, 2H, J=8.4 Hz)

¹³C-NMR (DMSO-d₆, 400 MHz) δppm: 28.6, 34.9, 48.4, 54.9, 73.1, 77.8, 115.1, 129.3, 130.3, 155.5, 155.9

mass spectrum m/e: 314 (M−H)⁻

### Example 2

### Production of (2S,3S)-N-tert-butoxycarbonyl-3-amino-1,2-epoxy-4-(4-hydroxyphenyl)butane

2-Propanol (38 ml) was added to (2S,3S)-N-tert-butoxycarbonyl-3-amino-4-(4-benzyloxyphenyl)-1-chloro-2-hydroxybutane (3.83 g) obtained in the same manner as in Reference Example 6.20% palladium hydroxide on carbon (0.192 g, manufactured by Kawaken Fine Chemicals Co., Ltd., moisture content 49.1%) was further added under an argon atmosphere, and the mixture was stirred under a hydrogen atmosphere (1 atm) at 60° C. for 1.5 hr. This reaction mixture was allowed to cool to 20° C. and 6N aqueous sodium hydroxide solution (2.93 ml) was added. Palladium hydroxide on carbon was removed by filtration, and the filtered palladium hydroxide on carbon was washed with a mixed solution of 2-propanol (7.6 ml) and 6N aqueous sodium hydroxide solution (1.47 ml). The palladium hydroxide on carbon was removed by filtration, and the obtained mother liquor and the washing liquid were combined and stirred at 20° C. for 2 hr. 6N Aqueous sodium hydroxide solution (0.146 ml) was added, and the mixture was stirred for 1 hr. Water (41.4 ml) and citric acid (1.77 g) were added to the reaction mixture to quench the reaction. This solution was cooled to 0° C. over 3 hr and further stirred at 0° C. for 7 hr. The obtained crystals were collected by filtration and washed with a mixed solution of 2-propanol (3.8 ml) and water (11.5 ml). The crystals were washed twice with water (38 ml, 38 ml). The obtained crystals were dried under reduced pressure to give the object (2S,3S)-N-tert-butoxycarbonyl-3-amino-1,2-epoxy-4-(4-hydroxyphenyl)butane (2.39 g, yield 91%). Chiral analysis by HPLC confirmed an optical purity to be not less than 99.5%.

¹H-NMR (DMSO-d₆,400 MHz) δppm: 1.30 (s, 9H), 2.54-2.68 (m, 3H), 2.73 (dd, 1H, J=14.0, 4.4 Hz), 2.85-2.89 (m, 1H), 3.37-3.45 (m, 1H), 6.64 (d, 2H, J=8.4 Hz), 6.78 (d, 1H, J=8.8 Hz), 6.98 (d, 2H, J=8.4 Hz), 9.14 (s, 1H)

¹³C-NMR (DMSO-d₆, 400 MHz) δppm: 28.5, 36.6, 44.9, 53.4, 53.6, 78.0, 115.2, 128.7, 130.3, 155.6, 155.9

mass spectrum m/e: 280 (M+H)⁺

[α]²⁰_{D}=−3.8° (c=1.0, MeOH)

melting point: 160-161° C.

### Example 3

### Production of (2R,3R)-N-tert-butoxycarbonyl-3-amino-1,2-epoxy-4-(4-hydroxyphenyl)butane

2-Propanol (17 ml) was added to (2R,3R)-N-tert-butoxycarbonyl-3-amino-4-(4-benzyloxyphenyl)-1-chloro-2-hydroxybutane (1.70 g) obtained in the same manner as in Reference Example 7. 20% palladium hydroxide on carbon (0.085 g, manufactured by Kawaken Fine Chemicals Co., Ltd., moisture content 49.1%) was further added under an argon atmosphere, and the mixture was stirred under a hydrogen atmosphere (1 atm) at 60° C. for 2 hr. This reaction mixture was allowed to cool to 20° C., and 6N aqueous sodium hydroxide solution (1.40 ml) was added. Palladium hydroxide on carbon was removed by filtration, and the filtered palladium hydroxide on carbon was washed with a mixed solution of 2-propanol (3.4 ml) and 6N aqueous sodium hydroxide solution (0.698 ml). The palladium hydroxide on carbon was removed by filtration, and the obtained mother liquor and the washing liquid were combined and stirred at 20° C. for 1.5 hr. Water (18.3 ml) and citric acid (0.805 g) were added to the reaction mixture to quench the reaction. This solution was cooled to 0° C. over 3 hr and further stirred at 0° C. for 12 hr. The obtained crystals were collected by filtration and washed with a mixed solution of 2-propanol (1.7 ml) and water (5.1 ml). The crystals were washed twice with water (17 ml, 17 ml). The obtained crystals were dried under reduced pressure to give the object (2R,3R)-N-tert-butoxycarbonyl-3-amino-1,2-epoxy-4-(4-hydroxyphenyl)butane (1.07 g, yield 91%).

### Reference Example 8

### Production of (2S,3S)-N-tert-butoxycarbonyl-3-amino-4-(4-benzyloxyphenyl)-1-chloro-2-hydroxybutane

The mother liquor resulting from the collection of the crystals by filtration in Reference Example 5 was analyzed to find that 2.97 g of N-tert-butoxycarbonyl-3-amino-4-(4-benzyloxyphenyl)-1-chloro-2-hydroxybutane ((2S,3S)/(2R,3S)=42.0/58.0) was contained therein. This solution was concentrated under reduced pressure. Water (50 ml) was added to the obtained crystals, and the mixture was stirred at room temperature for 1 hr. The crystals were collected by filtration. A mixed solution of hexane (7.5 ml) and ethyl acetate (7.5 ml) was added to the crystals, and the mixture was stirred at room temperature for 1 hr. The crystals were collected by filtration and dried under reduced pressure to give (2S,3S)-N-tert-butoxycarbonyl-3-amino-4-(4-benzyloxyphenyl)-1-chloro-2-hydroxybutane (1.05 g, recovery yield 35.3%, (2S,3S)/(2R,3S)=84.5/15.5).

### Example 4

### Production of (2S,3S)-N-tert-butoxycarbonyl-3-amino-1,2-epoxy-4-(4-hydroxyphenyl)butane

2-Propanol (9.2 ml) and 5% aqueous sodium hydrogen carbonate solution (0.379 mg) were added to (2S,3S)-N-tert-butoxycarbonyl-3-amino-4-(4-benzyloxyphenyl)-1-chloro-2-hydroxybutane (0.915 g, (2S,3S)/(2R,3S)=84.5/15.5) obtained in the same manner as in Reference Example 8.20% palladium hydroxide on carbon (0.046 g, manufactured by Kawaken Fine Chemicals Co., Ltd., moisture content 49.1%) was added under an argon atmosphere, and the mixture was stirred under a hydrogen atmosphere at 40° C. for 30 min. Palladium hydroxide on carbon was removed by filtration, and the filtered palladium hydroxide on carbon was washed with a mixed solution of 2-propanol (1.8 ml) and 6N aqueous sodium hydroxide solution (1.08 ml). The obtained reaction mixture was stirred at 20° C. for 2 hr. Water (9.4 ml) and citric acid (0.433 g) were added to this reaction mixture to quench the reaction. This reaction mixture was stirred at 0° C. for 1 hr, and the obtained crystals were collected by filtration. The crystals were washed with a mixed solution of 2-propanol (0.75 ml) and water (2.3 ml) and then further washed with water (9.2 ml, 9.2 ml). The obtained crystals were dried under reduced pressure to give the object (2S,3S)-N-tert-butoxycarbonyl-3-amino-1,2-epoxy-4-(4-hydroxyphenyl)butane (0.466 g, yield 74%, (2S,3S)/(2R,3S)=97.712.3).

### Comparative Example 1

2-Propanol (1.86 ml) and 6N aqueous sodium hydroxide solution (0.058 ml) were added to (2R,3R)-N-tert-butoxycarbonyl-3-amino-4-(4-benzyloxyphenyl)-1-chloro-2-hydroxybutane (93.4 g) obtained in the same manner as in Reference Example 7, and the mixture was stirred at 20° C. for 1 hr. Consumption of the starting material was confirmed, and citric acid (7.4 mg) was added to quench the reaction. 20% palladium hydroxide on carbon (4.7 mg, manufactured by Kawaken Fine Chemicals Co., Ltd., moisture content 49.1%) and water (0.560 ml) were added under an argon atmosphere, and the mixture was stirred under a hydrogen atmosphere at 60° C. After 1 hr, the reaction was analyzed by HPLC to confirm that debenzylation of the intermediate (2R,3R)-N-tert-butoxycarbonyl-3-amino-1,2-epoxy-4-(4-benzyloxyphenyl)butane had proceeded to give the object (2R,3R)-N-tert-butoxycarbonyl-3-amino-1,2-epoxy-4-(4-hydroxyphenyl)butane (76.2% by area). After further stirring for 16 hr, the reaction was analyzed by HPLC to find that the object (2R,3R)-N-tert-butoxycarbonyl-3-amino-1,2-epoxy-4-(4-hydroxyphenyl)butane was 9.5% by area, thus confirming a number of impurities. The results reveal that debenzylation of epoxy compound after synthesis makes control of debenzylation reaction difficult.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for making an aminoepoxide compound represented by formula (1): 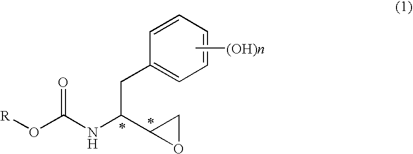
wherein R is an alkyl group or a fluorenylmethyl group, n is an integer of 1 or 2, and * indicates an asymmetric carbon atom independently having an R or S configuration,
wherein said method comprises:
(a) hydrogenating a compound represented by formula (2): 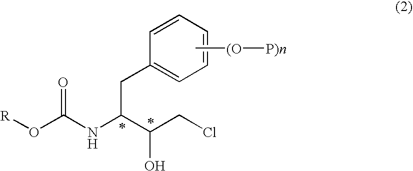
wherein R, n, and * are as defined above and P is a benzyl group optionally having one or more substituents, in the presence of a metal catalyst to obtain a chlorohydrin compound represented by formula (3) 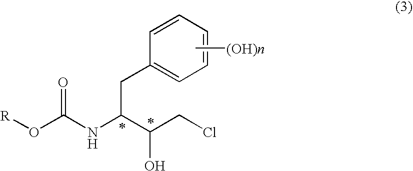
wherein R, n, and * are as defined above; and
(b) treating said chlorohydrin compound of formula (3) with a base, to obtain said compound of formula (1).

2. The method of claim 1, wherein configurations of the 2-position and the 3-position of said aminoepoxide compound represented by formula (1), said compound represented by formula (2) and said chlorohydrin compound represented by formula (3) are each an S configuration or an R configuration.

3. The method of claim 1, wherein R is a tert-butoxycarbonyl group, n is 1, and the position of substitution on the aromatic ring is the 4-position.

4. The method of claim 1, wherein said compound of formula (1) is (2S,3S)-N-tert-butoxycarbonyl-3-amino-1,2-epoxy-4-(4-hydroxyphenyl)butane.

5. The method of claim 1, wherein said compound of formula (1) is (2R,3R)-N-tert-butoxycarbonyl-3-amino-1,2-epoxy-4-(4-hydroxyphenyl)butane.

6. The method of claim 1, wherein said metal catalyst comprises at least one catalyst selected from the group consisting of palladium hydroxide on carbon, palladium carbon, Lindlar's catalyst, and mixtures thereof.

7. The method of claim 1, wherein said metal catalyst comprises palladium hydroxide on carbon.

8. The method of claim 1, wherein said hydrogenating is carried out in a solvent, and wherein said solvent comprises at least one solvent selected from the group consisting of a protic solvent, an aprotic solvent, and mixtures thereof.

9. The method of claim 8, wherein said solvent comprises at least one solvent selected from the group consisting of methanol, ethanol, 1-propanol, 2-propanol, 1-butanol, water, and mixtures thereof.

10. The method of claim 8, wherein said solvent comprises at least one solvent selected from the group consisting of ethyl acetate, isopropyl acetate, acetone, 2-butanone, methylisobutylketone, tetrahydrofuran, 1,4-dioxane, toluene, and mixtures thereof.

11. The method of claim 8, wherein said solvent comprises a solvent selected from the group consisting of 2-propanol and a mixed solvent of 2-propanol and water.

12. The method of claim 8, wherein said solvent comprises a mixed solvent of 2-propanol and water and wherein the volume ratio of 2-propanol:water is in the range of 1:1 to 100:1.

13. The method of claim 8, wherein said solvent comprises a mixed solvent of 2-propanol and water and wherein the volume ratio of 2-propanol:water is in the range of 1:1 to 50:1.

14. The method of claim 1, wherein said base comprises at least one base selected from the group consisting of aqueous lithium hydroxide solution, aqueous sodium hydroxide solution, aqueous potassium hydroxide solution, aqueous sodium carbonate solution, aqueous potassium carbonate solution, aqueous sodium hydrogen carbonate solution, aqueous potassium hydrogen carbonate solution, and mixtures thereof.

15. The method of claim 1, wherein said base comprises aqueous sodium hydroxide solution.

16. The method of claim 1, wherein said treating said chlorohydrin compound of formula (3) with a base is carried out in a solvent, and wherein said solvent comprises at least one solvent selected from the group consisting of methanol, ethanol, 1-propanol, 2-propanol, 1-butanol, water, and mixtures thereof.

17. The method of claim 16, wherein said solvent comprises a solvent selected from the group consisting of 2-propanol and a mixed solvent of 2-propanol and water.

18. The method of claim 16, wherein said solvent comprises a mixed solvent of 2-propanol and water and wherein the volume ratio of 2-propanol:water is in the range of 1:1 to 100:1.

19. The method of claim 16, wherein said solvent comprises a mixed solvent of 2-propanol and water and wherein the volume ratio of 2-propanol:water is in the range of 1:1 to 50:1.

20. The method of claim 1, wherein said hydrogenating and said treating said chlorohydrin compound of formula (3) with a base are both carried out in the same solvent and said solvent comprises a solvent selected from the group consisting of 2-propanol and a mixed solvent of 2-propanol and water.
